(19) United States
(12) Patent Application Publication
(10) Pub. No.: US 2013/0345506 A1
(43) Pub. Date: Dec. 26, 2013

(54) MAGNETIC-CONTROLLED SYSTEM APPLICABLE FOR COLONOSCOPY

(71) Applicants: Gi-Shih LIEN, Taipei (TW); Chih-Wen Liu, Taipei (TW); Joe-Air Jiang, Taipei (TW); Cheng-Long Chuang, Taipei (TW)

(72) Inventors: Gi-Shih LIEN, Taipei (TW); Chih-Wen Liu, Taipei (TW); Joe-Air Jiang, Taipei (TW); Cheng-Long Chuang, Taipei (TW)

(21) Appl. No.: 13/599,075

(22) Filed: Aug. 30, 2012

(30) Foreign Application Priority Data
Jun. 25, 2012 (TW) 101122597

(51) Int. Cl. A61B 1/01; A61B 1/31; A61B 1/05

(52) U.S. Cl. 600/109

(57) ABSTRACT

A magnetic-controlled system for colonoscopy is provided, including a supporting rod, an image retrieving unit, annular sheaths and a controlling device. After the supporting rod enters the large intestine of an organism via the anus, the image retrieving unit at the top end segment of the supporting rod then retrieves images of the large intestine. The annular sheaths are disposed around the supporting rod. Each of the annular sheaths has a plurality of magnetic-driven parts. The controlling device has a rotatable magnetic driving part. The magnetic-controlled system allows the rotation of the magnetic-driven parts, which in turn rotates the annular sheaths to facilitate the supporting rod moving smoothly in the large intestine, by rotating the magnetic driving parts and forward moving of the controlling device. Therefore, the pain experienced by the patients when the supporting rod is moving ungainly inside the intestine is greatly relieved.

### BACKGROUND OF THE INVENTION

1. Field of the Invention

The present invention relates to endoscopes, and, more particularly, to a magnetic-controlled system applicable for colonoscopy.

2. Description of Related Art

An endoscope is usually a specially-designed tube, the front end of which is a flexible end consisting mainly of a photographic device and a light source. When the photographic device is connected to a display, a user may place the endoscope into the interior of an organism, and the internal structure of the body can be displayed on the screen. For example, a throat endoscope (for examining the vocal cords, throat, and the like) is inserted through the nose; an upper gastrointestinal endoscope (for examining the esophagus, stomach and duodenum) is inserted through the mouth; and a colonoscope is inserted through the anus.

Colonoscopic inspection involves inserting a flexible tube with optic fibers from the anus into the intestine and visually examining the rectum and colon by progressively pushing the colonoscope through the intestine.

Since the large intestine is long and curved rather than straight inside the organism, and apart from the rectum, the descending colon and the ascending colon of the organism that are held in place by peritoneal, the other segments of the large intestine can move freely, so when the colonoscope encounters a bend or reaches deeper, the patient will feel the pain as the organs stretched to form a loop, making it difficult to carry out the inspection smoothly. Some patents may generate a sense of rejection about the colonoscopy, while some may experience other serious complications such as bleeding or perforation.

However, colonoscopy at present still offers best diagnosis for colonic etiologies and is more commonly adopted for gastrointestinal examination. In view of this, how to reduce the pain caused by colonoscopy in vivo manupulation in order to facilitate the examination has become an important and urgent issue to be solved.

### SUMMARY OF THE INVENTION

An objective of the present invention is to provide a magnetic-controlled system for colonoscopy that lessens the pain induced by a supporting rod moving inside the large intestine of the organism by changing the way in which the supporting rod of the colonoscope is controlled.

The magnetic-controlled system for colonoscopy of the present invention includes: a supporting rod including a flexible end for entering into the large intestine of a organism via the anus; an image retrieving unit fixed at the flexible end of the supporting rod for retrieving images of the large intestine when the supporting rod is in the organism; one or more annular sheaths including a plurality of magnetic-driven parts disposed around the supporting rod; and a controlling device including a magnetic driving part for actuating the magnetic-driven parts of the one or more annular sheaths by the magnetic driving part using magnetism, so that the annular sheath correspondingly rotates and moves inside the organism along with the actuation of the magnetic-driven parts.

From the above, the present invention uses the magnetic driving parts of the controlling device to correspondingly actuate the magnetic-driven parts of the annular sheaths, so that the annular sheaths can rotate and be fixed or pulled within the large intestine by the controlling device via magnetic rotation and pulling. This directly controls the movement of the front end of the supporting rod in the large intestine of the organism, avoiding damage caused by looping of the supporting rod in the rectum or the sigmoid colon when the supporting rod is manually pushed into the organism in the prior art during the colonoscopy examination. In addition, the driving device of the present invention rotates the magnetic driving part, which in turn rotates the magnetic-driven parts of the annular sheaths. Thread-like spiral grooves provided on the surface of the annular sheaths therefore rubs against the large intestine to facilitate the movement of the supporting rod and the image retrieving unit inside the large intestine, helping the supporting rod to pass through the obstacle of the colon haustra, and reducing the pain of the patients caused by the supporting rod moving ungainly in the large intestine as encountered in the prior art.

### BRIEF DESCRIPTION OF THE DRAWINGS

The present invention can be more fully understood by reading the following detailed description of the preferred embodiments, with reference made to the accompanying drawings, wherein:

FIG. 1 is a schematic diagram illustrating a magnetic-controlled system for colonoscopy 1 according to the present invention;

FIG. 2 is a schematic diagram illustrating magnetic poles arrangement of one annular sheath;

FIG. 3 is a schematic diagram illustrating a thread-like spiral groove on the annular sheath; and

FIG. 4 is a schematic diagram illustrating an embodiment of the present invention.

### DETAILED DESCRIPTION OF THE EMBODIMENTS

The present invention is described by the following specific embodiments. Those with ordinary skills in the arts can readily understand the other advantages and functions of the present invention after reading the disclosure of this specification. The present invention can also be implemented with different embodiments. Various details described in this specification can be modified based on different viewpoints and applications without departing from the scope of the present invention.

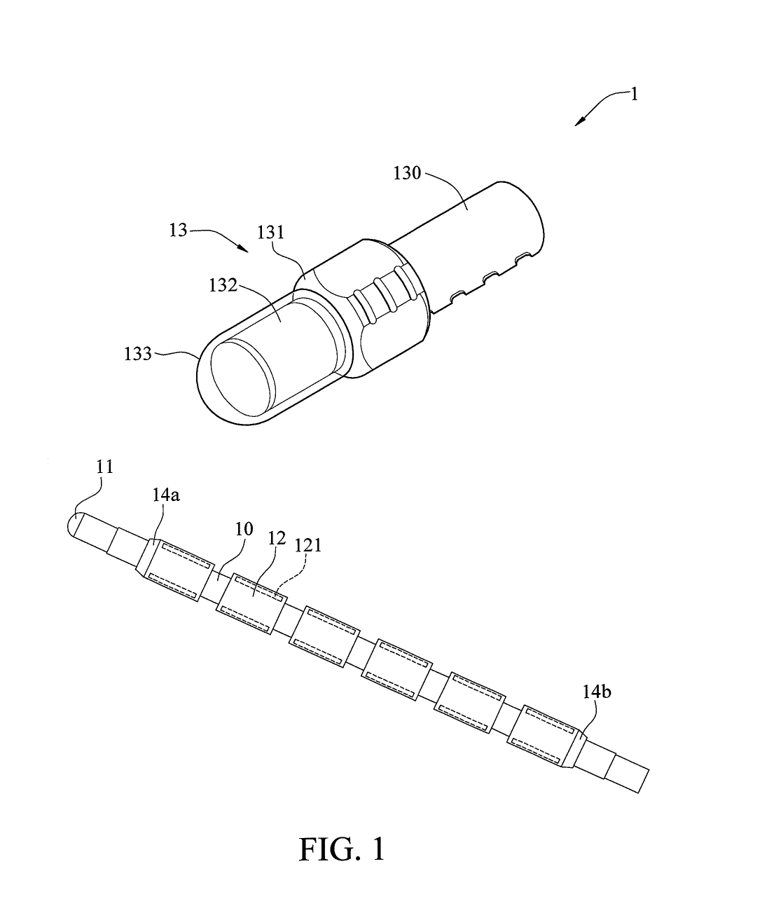

Referring to FIG. 1, a schematic diagram illustrating a magnetic-controlled system for colonoscopy 1 according to the present invention is shown. The magnetic-controlled system for colonoscopy 1 of the present invention includes a supporting rod 10, an image retrieving unit 11, annular sheaths 12 and a controlling device 13. The supporting rod 10 is an uncontrollable part at the front end of the colonoscope; only the top end segment of the supporting rod 10 is flexible for inserting into an organism through the anus. The image retrieving unit 11 is fixed at the top end segment of the supporting rod 10 for retrieving images inside the large intestine when the supporting rod 10 is in the organism. The annular sheath 12 is encased around the outer surface of the supporting rod 10, and a plurality of magnetic-driven parts 121 is disposed therein (or thereon). These magnetic-driven parts 121 may rotate when the annular sheath 12 is magnetically controlled.

Furthermore, positioning parts 14a and 14b are disposed on the supporting rod 10. The positioning parts 14a and 14b are made of silicone or soft materials. They are located at either end of the annular sheaths 12 for holding the annular sheaths 12 in place on the supporting rod 10, preventing displacement and detachment of the annular sheaths 12 from the supporting rod 10 during rotation and pulling controlled by the controlling device 13.

The controlling device 13 includes a rod 130, a magnetic driving part 132 and a cover 133. The rod 130 has a top end 131. The magnetic driving part 132 and the cover 133 are disposed at the top end 131 of the rod 130. The cover 133 envelops the magnetic driving part 132.

In the magnetic-controlled system for colonoscopy 1 of the present invention, the magnetic driving part 132 of the controlling device 13 magnetically controls the magnetic-driven parts 121 of the annular sheath 12, so that the magnetic-driven parts 121 rotate with the magnetic driving part 132 when under control of the magnetic driving part 132, which facilitates in vivo movement of the supporting rod 10 in the large intestine.

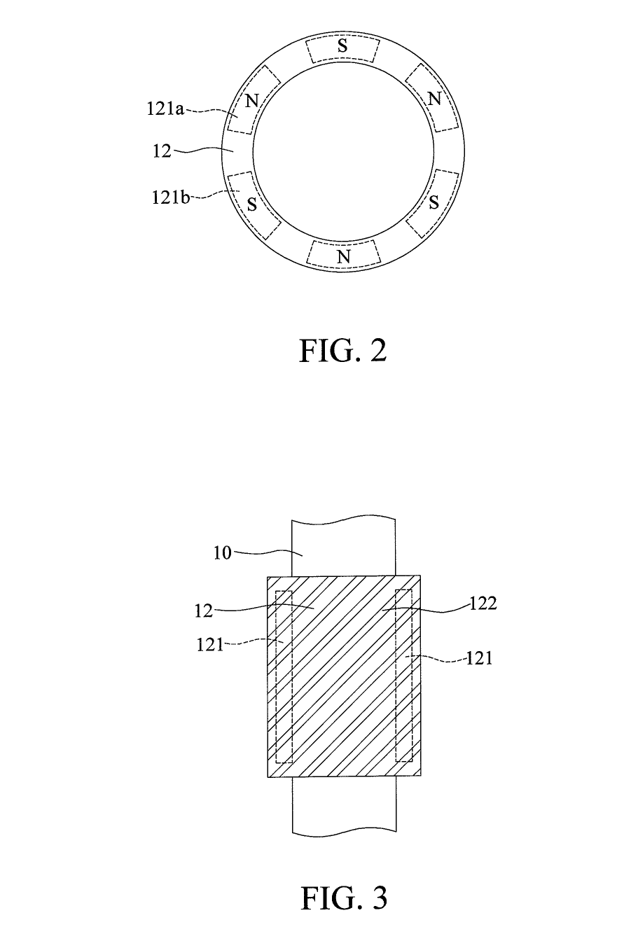

Referring to FIG. 2, a schematic diagram illustrating magnetic poles arrangement of one annular sheath 12 is shown. The annular sheath 12 includes a plurality of magnetic-driven parts 121, and each of the magnetic-driven parts 121 has an N pole 121a and an S pole 121b. The magnetic-driven parts can be embedded within the annular sheath 12 (that is, the magnetic-driven parts are coated by the annular sheath) or disposed on the surface of the annular sheath. In order to clearly define the distribution of the magnetic-driven parts 121 of the annular sheath 12, the top end segment of the supporting rod 10 where the image retrieving unit 11 is located is defined as the top herein. In the drawing, the annular sheath 12 has a plurality of magnetic-driven parts 121, and each of the magnetic-driven parts 121 has an N pole 121a and an S pole 121b. The magnetic-driven parts 121 are arranged in such a way that any two arbitrary adjacent magnetic-driven parts 121 have opposite polarities to each other. For example, for an arbitrary magnetic-driven part 121, if this magnetic-driven part 121 is disposed in the annular sheath 12 so that its S pole 121b is at the top, then the poles of the magnetic-driven parts 121 at either side of this magnetic-driven part 121 at the top are N poles 121a opposite to the S pole 121b. In other words, when an arbitrary magnetic-driven part 121 is disposed in the annular sheath 12 so that its S pole 121b at the top, the poles at either side of this S pole 121b are N poles 121a.

In addition, in order for the annular sheaths 12 to rotate under the control of the controlling device 13, the plurality of magnetic-driven parts 121 are distributed in/on the annular sheaths 12. Although six magnetic-driven parts 121 distributed in an annular sheath 12 are shown in the diagram, this number of magnetic-driven parts 121 is only for illustrative purpose, and the present invention is not so limited. In actual implementations, any number is possible as long as there is a plurality of magnetic-driven parts 121, though an even number of magnetic-driven parts is preferred. Similarly, the configuration of the N poles 121a and S poles 121b of the magnetic-driven parts 121 shown is for illustrative purpose only, and the present invention is not limited thereto.

Referring to FIG. 3, a schematic diagram illustrating a thread-like spiral groove 122 on the annular sheath is shown. As shown, the thread-like spiral groove 122 is provided on the magnetic-controlled system for colonoscopy 1 of the present invention. The thread-like spiral groove 122 is at an angle with respect to the supporting rod 10. When the annular sheaths 12 are pulled with rotation under the control of the controlling device 13, the annular sheaths 12 move the image retrieving unit 11 forward or backward in the intestine by rubbing the thread-like spiral groove 122 clockwise or counter-clockwise against the walls of the intestine folds.

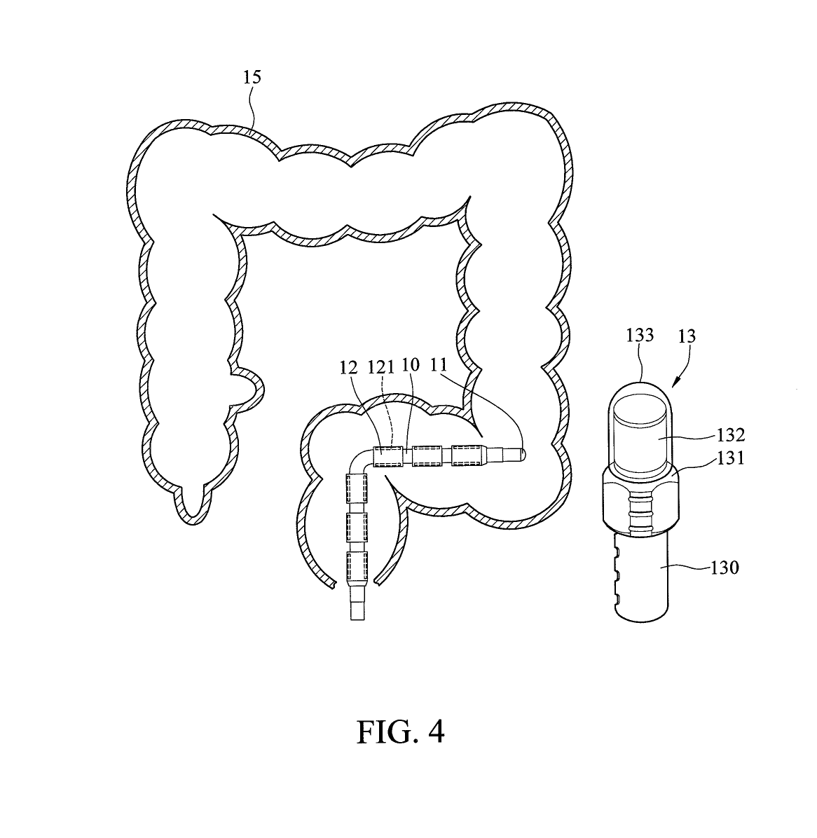

Referring to FIG. 4, a schematic diagram illustrating an embodiment of the present invention is shown. In an actual implementation of the magnetic-controlled system for colonoscopy 1 of the present invention, the supporting rod 10 and the image retrieving unit 11 enter into the large intestine 15 of an organism (not shown) through the anus, and the image retrieving unit 11 retrieves images inside the large intestine 15. When a portion of the supporting rod 10 enters into the large intestine 15, the magnetic-driven parts 121 of the annular sheaths 12 are correspondingly controlled by magnetism by the magnetic driving part 132 of the controlling device 13, so that the controlling device 13 may control the movement of the supporting rod 10 in the large intestine 15.

In practice, when a user attempts to move the supporting rod 10 along the large intestine 15, the bends in the large intestine 15 may prevent the supporting rod 10 from moving smoothly, and may make difficult loops on insertion by pushing the colonoscope. When this happens, the annular sheaths 12 are magnetically fixed to release the loops and then controlled to rotate with pulling by the controlling device 13, which in turn moves the supporting rod 10 forward in the large intestine 15, so that the supporting rod 10 can move smoothly in the large intestine 15.

A driving unit (not shown) is provided in the controlling device 13. The magnetic driving part 132 is coupled to and driven by the driving unit. In an embodiment, a stepper motor is used as the driving unit. When the supporting rod 10 encounters a bend in the large intestine 15 and cannot move smoothly, the supporting rod 10 can be bent under external control to pass through the bend in the large intestine 15. After the top end segment of the supporting rod 10 has passed through the bend of the large intestine 15, the driving unit of the controlling device 13 drives the magnetic driving part 132 to rotate, which in turn rotates the annular sheaths 12; meanwhile, the controlling device 13 moves forward again, enabling the supporting rod 10 to pass through the bend of the large intestine 15 smoothly by pulling. As a result, the supporting rod 10 is moving in the large intestine 15 not only by pushing but also by pulling the supporting rod 10. In addition, the thread-like spiral groove 122 is provided on each of the annular sheaths 12 and is in contact with the walls of the folds of the large intestine 15, so when the controlling device 13 controls the movement of the supporting rod 10 and the image retrieving unit 11, the annular sheaths 12 move the image retrieving unit 11 forward or backward in the large intestine 15 by rubbing the thread-like spiral grooves 122 against the walls of the intestine folds clockwise or counter-clockwise. The pain that patients may experience is relieved when the supporting rod 10 is moving inside their bodies without making difficult loops. For ease of understanding and a clear illustration, the thread-like spiral grooves 122 are not shown in FIG. 4.

The above embodiments are only used to illustrate the principles of the present invention, and they should not be construed as to limit the present invention in any way. The above embodiments can be modified by those with ordinary skill in the art without departing from the scope of the present invention as defined in the following appended claims.

What is claimed is:

1. A magnetic-controlled system for colonoscopy, comprising:
a supporting rod including a flexible end for entering into a large intestine of an organism via an anus;
an image retrieving unit fixed at the flexible end of the supporting rod for retrieving images of the large intestine when the supporting rod is in the organism;
one or more annular sheaths including a plurality of magnetic-driven parts disposed around the supporting rod; and
a controlling device including a magnetic driving part for actuating the magnetic-driven parts of the annular sheaths by the magnetic driving part using magnetism, so that the annular sheath correspondingly fixes or moves inside the organism along with the actuation of the magnetic-driven parts.

2. The magnetic-controlled system of claim 1, wherein a thread-like spiral groove is provided on the surface of each of the one or more annular sheaths, and, when the controlling device controls to rotate the annular sheaths, and to move the image retrieving unit forward or backward in the large intestine by rubbing the thread-like spiral grooves against the walls of the folds of the large intestine clockwise or counter-clockwise.

3. The magnetic-controlled system of claim 2, wherein the thread-like spiral grooves are at an angle with respect to the supporting rod.

4. The magnetic-controlled system of claim 1, wherein each of the plurality of magnetic-driven parts includes an N pole and an S pole, and two adjacent magnetic-driven parts have opposite polarities.

5. The magnetic-controlled system of claim 1, wherein positioning parts are provided on the supporting rod on either side of the annular sheaths for holding the annular sheaths in place on the supporting rod.

6. The magnetic-controlled system of claim 5, wherein the positioning parts are made of silicon.

7. The magnetic-controlled system of claim 1, wherein the controlling device further comprises a rod with a top end, a driving unit disposed in the rod and a cover for covering the magnetic driving parts, and the magnetic driving parts are disposed at the top end of the rod and coupled to the driving unit to be driven into rotation by the driving unit.

8. The magnetic-controlled system of claim 7, wherein the driving unit includes a stepper motor.

9. The magnetic-controlled system of claim 1, wherein the magnetic-driven parts are provided underneath the surface of the one or more annular sheaths and embedded into the one or more annular sheaths.
